(19) United States
(12) Patent Application Publication
(10) Pub. No.: US 2024/0087745 A1
(43) Pub. Date: Mar. 14, 2024

(54) METHOD FOR ASSESSMENT OF VIRUS INFECTION RISK AND PORTABLE DETECTOR THEREOF

(71) Applicant: Tien-Tse Hsieh, Tainan City (TW)

(72) Inventor: Tien-Tse Hsieh, Tainan City (TW)

(21) Appl. No.: 18/465,974

(22) Filed: Sep. 12, 2023

Related U.S. Application Data
(60) Provisional application No. 63/405,929, filed on Sep. 13, 2022.

(51) Int. Cl. G16H 50/20; G01N 33/00; G16H 50/30; G16H 50/80

(52) U.S. Cl. CPC G16H 50/20; G01N 33/004; G16H 50/30; G16H 50/80

(57) ABSTRACT

A method for assessment of a virus infection risk and a portable detector thereof are disclosed. The portable detector is used to perform the following steps: obtaining and analyzing at least one control CO₂ value and at least one test CO₂ value to obtain at least one CO₂ impact value; obtaining and analyzing at least one control PM2.5 value and at least one test PM2.5 value to obtain at least one PM2.5 impact value; analyzing the CO₂ impact value and PM2.5 impact value to obtain at least one human activity factor. When the human activity factor is less than a set value, it is determined as a low virus infection risk; when the human activity factor is greater than the set value, it is determined as a high virus infection risk. The portable detector has the advantages of easy portability, no consumables and real-time warnings.

### CROSS REFERENCE TO RELATED APPLICATIONS

This application claims the benefit of U.S. Provisional Application No. 63/405,929, filed Sep. 13, 2022.

### FIELD OF THE INVENTION

The present invention relates to a method for assessment of a virus infection risk and a portable detector thereof, and more particularly to a portable detector having the advantages of easy portability, no consumables and real-time warnings.

### BACKGROUND OF THE INVENTION

Conventional assessment techniques of virus infection risks have many problems in practical applications. The problems may influence the efficiency and convenience of the assessment techniques.

First, these assessment techniques often require a lot of time. For example, fluorescence analysis is used for detection. This is time-consuming and cumbersome. These assessment techniques also require a large amount of consumables, such as chemicals or filters, which not only increases costs but also places an unnecessary burden on the environment. Another disadvantage of the conventional assessment techniques is that the cost of the equipment is high. Many assessment techniques require expensive equipment and devices, which may be a burden for many laboratories and institutions.

The conventional assessment techniques are difficult to achieve consistent results quickly when performing virus risk detections in different locations. They cannot immediately alert the public to take corresponding protective measures, so it is impossible to reduce the risk of infection effectively. The conventional assessment techniques have many shortcomings. There is a need for a new technique to overcome these problems and improve the efficiency and reliability of detection.

Accordingly, the inventor of the present invention has devoted himself based on his many years of practical experiences to solve these problems.

### SUMMARY OF THE INVENTION

In view of the foregoing deficiencies of the existing technology, the technical problem to be solved by the present invention is to provide a method for assessment of a virus infection risk and a portable detector thereof.

According to one aspect of the present invention, a method for assessment of a virus infection risk is provided. The method comprises the following steps: obtaining at least one control CO₂ value and at least one test CO₂ value through a receiving module, and analyzing the control CO₂ value and the test CO₂ value through a computation module to obtain at least one CO₂ impact value; obtaining at least one control PM2.5 value and at least one test PM2.5 value through the receiving module, and analyzing the control PM2.5 value and the test PM2.5 value through the computation module to obtain at least one PM2.5 impact value; analyzing the CO₂ impact value and PM2.5 impact value through the computation module to obtain at least one human activity factor. When the human activity factor is less than a set value, it is determined as a low virus infection risk by the computation module; when the human activity factor is greater than the set value, it is determined as a high virus infection risk by the computation module, and a warning module issues a warning.

The control CO₂ value and the control PM2.5 value are the values measured in a first environment. The test CO₂ value and the test PM2.5 value are the values measured in a second environment.

When the test CO₂ value is twice or more than twice the control CO₂ value, the computation module automatically determines that the location of the receiving module changes from the relatively open first environment (such as an outdoor space) to the relatively enclosed second environment (such as an indoor space). In this way, the warning module may alert the user to pay attention to ventilation, or the receiving module may increase the frequency of updating data collection.

The control CO₂ value and the control PM2.5 value are the values of the first environment received by a network. The test CO₂ value and the test PM2.5 value are the values measured in the second environment. The user may set the state of the second environment manually when entering an indoor space from an outdoor space.

The method further comprises obtaining at least one control PM10 value and at least one test PM10 value through the receiving module, and analyzing the control PM10 value and the test PM10 value through the computation module to obtain a PM10 impact value. When the PM2.5 impact value is greater than the PM10 impact value, it is determined as data distortion, and the PM10 impact value is used to analyze the human activity factor.

The method further comprises obtaining at least one test environment temperature and at least one test environment humidity through the receiving module. When the computation module determines that the human activity factor is the high virus infection risk, a virus species suitable for transmission at the test environment temperature and the test environment humidity is assessed, and the warning module issues the warning about the high virus infection risk and the virus species.

According to one aspect of the present invention a portable detector for assessment of a virus infection risk is provided. The portable detector comprises a receiving module, a computation module, and a warning module. The receiving module includes a CO₂ sensor, a PM2.5 sensor, a temperature sensor and a humidity sensor for measuring and obtaining at least one CO₂ impact value, at least one PM2.5 impact value, at least one test environment temperature and at least one test environment humidity. The computation module is connected to the receiving module. The computation module analyzes the CO₂ impact value and the PM2.5 impact value to obtain at least one human activity factor. When the human activity factor is determined to be a high virus infection risk, a virus species suitable for transmission at the test environment temperature and the test environment humidity is assessed. The warning module is connected to the computation module. The warning module issues a warning about the virus species that is assessed as the high virus infection risk by the computation module.

The receiving module further includes a PM10 sensor. The PM10 sensor measures and obtains at least one PM10 impact value. When the PM2.5 impact value is greater than the PM10 impact value, it is determined as data distortion, and the PM10 impact value is used to analyze the human activity factor.

The CO₂ impact value includes at least one control CO₂ value and at least one test CO₂ value. The PM2.5 impact value includes at least one control PM2.5 value and at least one test PM2.5 value. The control CO₂ value and the control PM2.5 value are data received or measured in a first environment. The test CO₂ value and the test PM2.5 value are data measured in a second environment. The second environment is different from the first environment. The first environment may be, for example, an open outdoor space. The second environment may be, for example, an enclosed or semi-open indoor space.

The receiving module further includes a wireless receiver. The wireless receiver receives the control CO₂ value and the control PM2.5 value of a local database.

The primary object of the present invention is to obtain the control CO₂ value, the test CO₂ value, the control PM2.5 value and the test PM2.5 value through the portable detector that is easy to carry, and then analyze and obtain the human activity factor. The human activity factor is used to determine whether the environment where the user is located has a high risk of virus infection, and a warning is issued. Further, the temperature and humidity of the environment is analyzed for the survival rate and transmission rate of various viruses, so as to accurately warn which viruses in the environment are high risk. The present invention has the advantages of easy portability, no consumables and real-time warning.

The invention as well as a preferred mode of use, further objectives and advantages thereof will be best understood by reference to the following detailed description of illustrative embodiments when read in conjunction with the accompanying drawings.

### BRIEF DESCRIPTION OF THE DRAWINGS

FIG. 1 is a schematic view showing the structure of a portable detector of the present invention;

FIG. 2 is a block diagram showing a method for assessment of a virus infection risk of the present invention; and

FIG. 3 is a flowchart of the method of the present invention.

### DETAILED DESCRIPTION OF THE PREFERRED EMBODIMENTS

For further illustrating the means and functions by which the present invention achieves the certain objectives, the following description, in conjunction with the accompanying drawings and preferred embodiments, is set forth as below to illustrate the implement, structure, features and effects of the subject matter of the present invention.

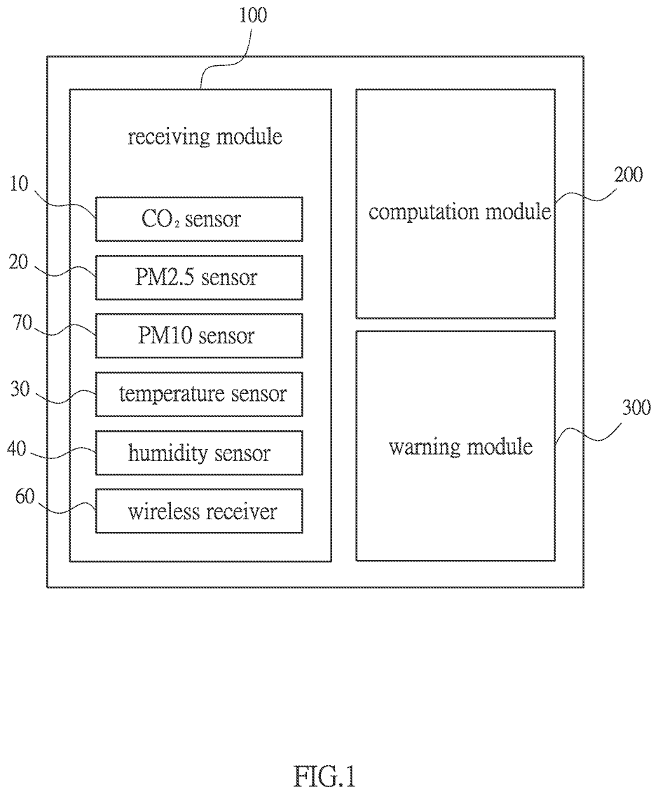

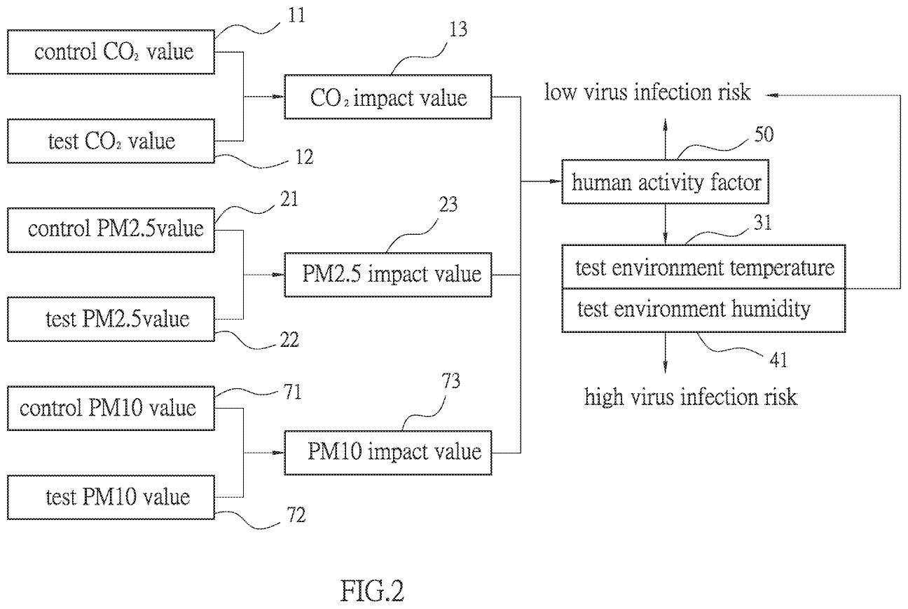

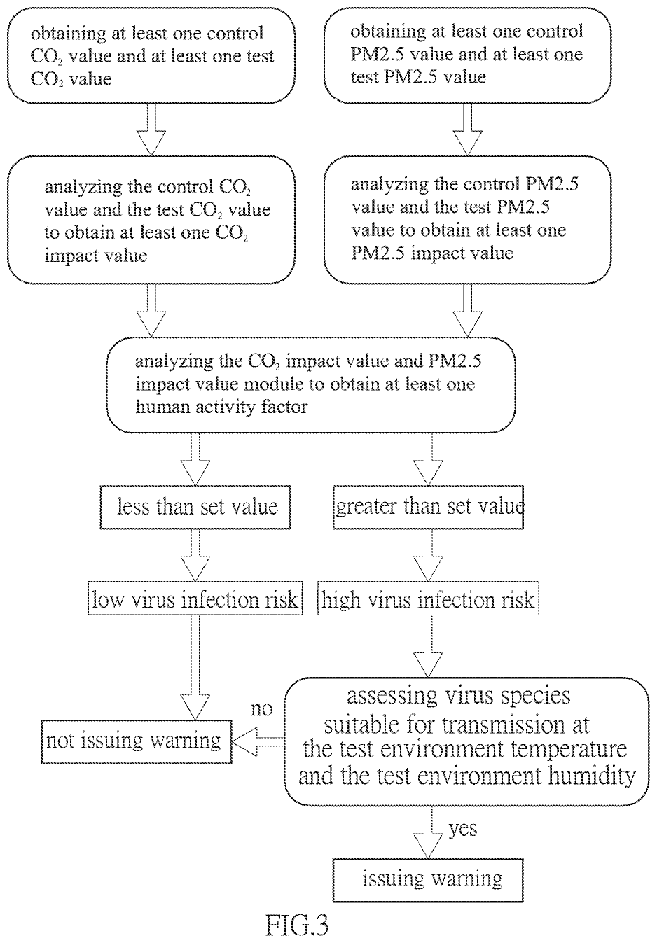

As shown in FIG. 1, FIG. 2 and FIG. 3, the present invention discloses a method for assessment of a virus infection risk and a portable detector thereof. The portable detector comprises a receiving module (100), a computation module (200), and a warning module (300).

The receiving module (100) includes a CO₂ sensor (10), a PM2.5 sensor (20), a temperature sensor (30) and a humidity sensor (40) for measuring and obtaining at least one CO₂ impact value (13), at least one PM2.5 impact value (23), at least one test environment temperature (31) and at least one test environment humidity (41). The CO₂ impact value (13) includes at least one control CO₂ value (11) and at least one test CO₂ value (12). The PM2.5 impact value (23) includes at least one control PM2.5 value (21) and at least one test PM2.5 value (22). The control CO₂ value (11) and the control PM2.5 value (21) are data received or measured in a first environment. The test CO₂ value (12) and the test PM2.5 value (22) are data measured in a second environment. The first environment is different from the second environment. The first environment may be, for example, an open outdoor space. The second environment may be, for example, an enclosed or semi-open indoor space.

The computation module (200) is connected to the receiving module (100), and analyzes the CO₂ impact value (13) and the PM2.5 impact value (23) to obtain at least one human activity factor (50). When the human activity factor (50) is determined to be a high virus infection risk, a virus species suitable for transmission at the test environment temperature (31) and the test environment humidity (41) is assessed.

The human activity factor (50) is used to determine the density of people in a specific space and the intensity of human activities. For example, a space with a large number of people is compared with a space with a small number of people, and the people in the space are moving or sitting still. The CO₂ concentration and particulate matter in the former will be higher than those in the latter. The human activity factor (50) may automatically generate a set value based on region, season, day and night and other conditions, and the user may adjust the set value based on space, the number of people in the space, or climate conditions. When the human activity factor (50) is higher than the set value, the portable detector will generate a warning signal of a high virus infection risk.

The warning module (300) is connected to the computation module (200). The warning module (300) issues a warning about the virus species that is assessed as a high virus infection risk by the computation module (200). The warning module (300) may be a display device, a warning light, a horn, and so on, for issuing a warning by means of any one or a combination of two or more of warning lights, warning sounds or warning images.

Furthermore, as shown in FIG. 1, FIG. 2 and FIG. 3, the receiving module (100) further includes a wireless receiver (60) and a PM10 sensor (70). The wireless receiver (60) receives the control CO₂ value (11) and the control PM2.5 value (21) of the local database (cloud database). The wireless receiver (60) may be connected to a device, such as a mobile phone, through Wi-Fi, Bluetooth, etc., for receiving the Internet or cloud database through the mobile network. When the wireless receiver (60) cannot be connected to the Internet, it can automatically switch to obtain the control CO₂ value (11) and the control PM2.5 value (21) from built-in database, or obtain the control CO₂ value (11) and the control PM2.5 value (21) by means of a field measuring method.

The PM10 sensor 70 measures and obtains at least one PM10 impact value (73). When the PM2.5 impact value (23) is greater than the PM10 impact value (73), it is determined as data distortion, and the PM10 impact value (73) is used to analyze the human activity factor (50). The PM10 sensor (70) obtains at least one control PM10 value (71) and at least one test PM10 value (72), and then analyzes the control PM10 value (71) and the test PM10 value (72) to obtain the PM10 impact value (73).

Furthermore, the PM2.5 sensor (20) is used as the primary determination basis, and the PM10 sensor (70) is used as the secondary determination basis. The information measured by the PM10 sensor (70) can prove whether the PM2.5 sensor (20) is faulty, thereby instructing the user to calibrate or repair it. When the concentration of particle matter measured by the PM2.5 sensor (20) is less than 5 μg/m³, the PM10 sensor (70) replaces the PM2.5 sensor (20) for performing a determination, thereby improving the accuracy of the human activity factor (50).

As shown in FIG. 3, a method for assessment of a virus infection risk provided by the present invention comprises the following steps:

Step 1: obtaining at least one control CO₂ value (11) and at least one test CO₂ value (12) through the receiving module (100), and analyzing the control CO₂ value (11) and the test CO₂ value (12) through the computation module (200) to obtain at least one CO₂ impact value (13); wherein the control CO₂ value (11) is the value measured in the first environment, and the test CO₂ value (12) is the value measured in the second environment.

For example, the CO₂ concentration in outdoor spaces will be significantly lower than that in indoor spaces. When the test CO₂ value (12) is twice or greater than twice the control CO₂ value (11), the computation module (200) automatically determines that the location of the receiving module (100) changes from the relatively open first environment (such as an outdoor space) to the relatively enclosed second environment (such as an indoor space). In this way, the warning module (300) may alert the user to pay attention to ventilation, or the receiving module (100) may increase the frequency of the updating data collection. Thus, the user can ensure real-time assessment of the virus infection risk anytime and anywhere, without a network communication. There is a significant difference value between the control CO₂ value (11) and the test CO₂ value (12). When the difference value is greater than the specified value, it is determined that the CO₂ impact value (13) is high impact. This is one of many analytical methods. The present invention is not limited to using a differential analysis to obtain the CO₂ impact value (13). For example, a ratio value may be used to obtain the CO₂ impact value (13).

Step 2: obtaining at least one control PM2.5 value (21) and at least one test PM2.5 value (22) through the receiving module (100), analyzing the control PM2.5 value (21) and the test PM2.5 value (22) through the computation module (200) to obtain at least one PM2.5 impact value (23); wherein the control PM2.5 value (21) is the value measured in the first environment, and the test PM2.5 value (22) is the value measured in the second environment.

In one embodiment, the control CO₂ value (11) and the control PM2.5 value (21) are the values of the first environment received by the network. The test CO₂ value (12) and the test PM2.5 value (22) are the values measured in the second environment. The user may set the state of the second environment manually when entering the indoor space from the outdoor space. For example, the concentration of particulate matter (PM value) in outdoor spaces will be significantly greater than in indoor spaces. When the test PM2.5 value (22) measured in the indoor space is greater than the control PM2.5 value (21), the PM2.5 impact value (23) is determined to be high impact. This is one of many analysis methods. The present invention is not limited to using a comparative method to obtain the PM2.5 impact value (23).

Step 3: analyzing the CO₂ impact value (13) and PM2.5 impact value (23) through the computation module (200) to obtain at least one human activity factor (50). When the human activity factor (50) is less than the set value, it is determined as a low virus infection risk; when the human activity factor (50) is greater than the set value, it is determined as a high virus infection risk.

The human activity factor (50) is defined as the density of people in a specific space and the intensity of human activities. When the CO₂ impact value (13) is relatively high impact, it may be defined that the number of people accommodated in the unit space is great, people are in close contact, and the chance of transmitting the virus via droplets, from the nose and mouth, is high. When the PM2.5 impact value (23) is relatively high impact, it may be defined that the concentration of particulate matter in the unit space is high. That is, when the air is not circulated, the amount of the virus suspended in the air increases, and the chance of transmitting the virus increases. In this way, it can be preliminarily determined that there is a high virus infection risk in the environment.

Specifically, the relationship between the CO₂ impact value (13), the PM2.5 impact value (23), and the human activity factor (50) is described below. The CO₂ impact value (13) and the PM2.5 impact value (23) are assessed, for example, by a difference value (that is, the difference value between the control value and the test value). It may be preset that the difference value of CO₂ between 0 and 400 ppm is determined as low impact, the difference value of CO₂ between 400 and 1200 ppm is determined as medium impact, and the difference value of CO₂ between 1200 and 1600 ppm is determined as high impact. It may be preset that the difference value of PM2.5 between 0 and 20 μg/m³ is low impact, the difference value of PM2.5 between 20 and 40 μg/m³ is medium impact, and the difference value of PM2.5 above 40 μg/m³ is high impact. Based on the following correspondence table of high, medium and low impacts of CO₂ and PM2.5, the human activity factor (50) is obtained.

Correspondence table of human activity factors:

| | CO₂ | CO₂ | CO₂ |
|---|---|---|---|
| | high impact | medium impact | low impact |
| PM2.5 high | high human | high human activity | medium human |
| impact | activity factor | factor | activity factor |
| PM2.5 medium | high human | medium human | low human |
| impact | activity factor | activity factor | activity factor |
| PM2.5 low | medium human | low human activity | low human |
| impact | activity factor | factor | activity factor |

Through the above-mentioned correspondence table of human activity factors, high or/and medium human activity factors (50) may be set as a high virus infection risk. However, it should be noted that this is one of many analysis methods, and the present invention is not limited to only using the above method to analyze the human activity factor (50).

Step 4: obtaining at least one test environment temperature (31) and at least one test environment humidity (41) through the receiving module (200). When the human activity factor (50) is determined to be a high virus infection risk, the virus species suitable for transmission at the test environment temperature (31) and the test environment humidity (41) is assessed. Because the survival rate and transmission speed of various viruses and bacteria are affected by temperature and humidity, measuring the temperature and humidity in the environment is able to issue a warning of a high virus infection risk for the virus species that is easily transmitted in the space.

Example 1: Covid-19 is highly transmissible (having a high infection risk) in a low temperature and dry environment. That is, when the environment temperature is higher than 30° C. and the relative humidity is between 45% and 60%, the transmission efficiency of Covid-19 is not good. When the human activity factor (50) is determined to be a high virus infection risk, a further analysis is performed. If the test environment temperature (31) is lower than 30° C. and the test environment humidity (41) is greater than 90%, it will be determined that there is a high virus infection risk of Covid-19 in the current environment, and the user will be warned immediately. On the contrary, when the human activity factor (50) is determined to be a high virus infection risk and the test environment temperature (31) is 38° C., it is determined that it is not easy to transmit Covid-19 and no warning is issued. The warning may be set as multiple warnings (such as a low risk warning, a medium risk warning, a high risk warning, etc.).

Example 2: Influenza is highly transmissible (having a high infection risk) in a low temperature and dry environment. That is, when the environment temperature is higher than 22° C. and the relative humidity is greater than 50%, the transmission efficiency of influenza is not good. When the virus infection risk is high, the environment temperature is lower than 22° C. and the environment humidity is lower than 50%, a warning signal will be issued for the current high risk of virus infection of influenza. When the human activity factor (50) is determined to be a low virus infection risk, there is no need to analyze the environment temperature and humidity. When the human activity factor (50) is determined to be a high virus infection risk, it is necessary to measure the test environment temperature (31) and the test environment humidity (41), and analyze the transmissibility of each virus and bacteria, and then issue a high-risk warning for each virus and bacteria. Further, in keeping with the current virus or disease information in the area, the determination criteria of the CO₂ impact value (13), the PM2.5 impact value (23) and the human activity factor (50) are automatically adjusted, so as to accurately and instantly obtain risk assessments of viral and bacterial infections. The portable detector has the advantages of easy portability, no consumables and real-time warnings.

Example 3: When a warning signal of a high virus infection risk is issued, the portable detector can transmit information to an external device via the Internet, Wi-Fi or Bluetooth, so that nearby users of the portable detector may learn about the virus infection risk through other users. Alternatively, the cloud database collects a large number of information of virus infection risk, analyzes the level of virus infection risk in various regions, and automatically issues notifications about the level of risk, so that the users of the portable detector in the area can take appropriate protective measures, and the information about the virus infection risk can be shared with the public, thereby avoiding going to spaces with a high virus infection risk.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A method for assessment of a virus infection risk, comprising the following steps:
obtaining at least one control CO₂ value and at least one test CO₂ value through a receiving module, and analyzing the control CO₂ value and the test CO₂ value through a computation module to obtain at least one CO₂ impact value;
obtaining at least one control PM2.5 value and at least one test PM2.5 value through the receiving module, and analyzing the control PM2.5 value and the test PM2.5 value through the computation module to obtain at least one PM2.5 impact value;
analyzing the CO₂ impact value and PM2.5 impact value through the computation module to obtain at least one human activity factor, wherein when the human activity factor is less than a set value, it is determined as a low virus infection risk; when the human activity factor is greater than the set value, it is determined as a high virus infection risk, and a warning module issues a warning.

2. The method as claimed in claim 1, wherein the control CO₂ value and the control PM2.5 value are the values measured in a first environment, the test CO₂ value and the test PM2.5 value are the values measured in a second environment, and the second environment is different from the first environment.

3. The method as claimed in claim 2, wherein when the test CO₂ value is twice or more than twice the control CO₂ value, the computation module automatically determines that the location of the receiving module changes from the relatively open first environment to the relatively enclosed second environment.

4. The method as claimed in claim 1, wherein the control CO₂ value and the control PM2.5 value are the values of a first environment received by a network, the test CO₂ value and the test PM2.5 value are the values measured in a second environment, and the second environment is different from the first environment.

5. The method as claimed in claim 1, further comprising obtaining at least one control PM10 value and at least one test PM10 value through the receiving module, and analyzing the control PM10 value and the test PM10 value through the computation module to obtain a PM10 impact value, wherein when the PM2.5 impact value is greater than the PM10 impact value, it is determined as data distortion, and the PM10 impact value is used to analyze the human activity factor.

6. The method as claimed in claim 1, further comprising obtaining at least one test environment temperature and at least one test environment humidity through the receiving module, wherein when the computation module determines that the human activity factor is the high virus infection risk, a virus species suitable for transmission at the test environment temperature and the test environment humidity is assessed, and the warning module issues the warning about the high virus infection risk and the virus species.

7. A portable detector for assessment of a virus infection risk, comprising:
a receiving module, including a CO₂ sensor, a PM2.5 sensor, a temperature sensor and a humidity sensor for measuring and obtaining at least one CO₂ impact value, at least one PM2.5 impact value, at least one test environment temperature and at least one test environment humidity;
a computation module, connected to the receiving module, the computation module analyzing the CO₂ impact value and the PM2.5 impact value to obtain at least one human activity factor, wherein when the human activity factor is determined to be a high virus infection risk, a virus species suitable for transmission at the test environment temperature and the test environment humidity is assessed; and
a warning module, connected to the computation module, the warning module issuing a warning about the virus species that is assessed as the high virus infection risk by the computation module.

8. The portable detector as claimed in claim 6, wherein the receiving module further includes a PM10 sensor, the PM10 sensor measures and obtains at least one PM10 impact value, when the PM2.5 impact value is greater than the PM10 impact value, it is determined as data distortion, and the PM10 impact value is used to analyze the human activity factor.

9. The portable detector as claimed in claim 7, wherein the CO₂ impact value includes at least one control CO₂ value and at least one test CO₂ value, the PM2.5 impact value includes at least one control PM2.5 value and at least one test PM2.5 value, the control CO₂ value and the control PM2.5 value are data received or measured in a first environment, the test CO₂ value and the test PM2.5 value are data measured in a second environment, and the second environment is different from the first environment.

10. The portable detector as claimed in claim 9, wherein the receiving module further includes a wireless receiver, and the wireless receiver receives the control CO₂ value and the control PM2.5 value of a local database.
